(19) United States
(12) Patent Application Publication
(10) Pub. No.: US 2023/0414952 A1
(43) Pub. Date: Dec. 28, 2023

(54) HEADER ASSEMBLY FOR AN IMPLANTABLE INTRACARDIAC DEVICE AND RESPECTIVE INTRACARDIAC DEVICE

(71) Applicant: BIOTRONIK SE & Co. KG, Berlin (DE)

(72) Inventors: Eric Austin, Portland, OR (US); Devan Hughes, Tualatin, OR (US); Jeffrey A. von Arx, Lake Oswego, OR (US)

(73) Assignee: BIOTRONIK SE & Co. KG, Berlin (DE)

(21) Appl. No.: 18/253,498

(22) PCT Filed: Nov. 24, 2021
(86) PCT No.: PCT/EP2021/082819

§ 371 Date: May 18, 2023

Related U.S. Application Data
(60) Provisional application No. 63/119,042, filed on Nov. 30, 2020.

(30) Foreign Application Priority Data
Feb. 26, 2021 (EP) 21159588.9

(51) Int. Cl. A61N 1/375

(52) U.S. Cl. CPC A61N 1/3756; A61N 1/37518

(57) ABSTRACT

Implantable intracardiac device and a header assembly for such device having at least one tine extending from a base ring, further comprising a spacer and a header cap, wherein the base ring of the anchoring device is conically formed. The inventive intracardiac device has small dimensions and provides, at the same time, a robust attachment mechanism.

### CROSS-REFERENCE TO RELATED APPLICATIONS

This application is the United States National Phase under 35 U.S.C. § 371 of PCT International Patent Application No. PCT/EP2021/082819, filed on Nov. 24, 2021, which claims the benefit of European Patent Application No. 21159588.9, filed on Feb. 26, 2021, and U.S. Provisional Patent Application No. 63/119,042, filed on Nov. 30, 2020, the disclosures of which are hereby incorporated by reference herein in their entireties.

### TECHNICAL FIELD

The present invention refers to an implantable intracardiac device, such as an implantable intracardiac pacemaker, and a header assembly therefor.

### BACKGROUND

Active or passive intracardiac medical devices (IMD), for example, implantable intracardiac pacemakers (also known as leadless pacemakers), are well known miniaturized medical devices which are entirely implanted into a heart's chamber, e.g., ventricle or atrium. Intracardiac pacemakers are used for patients who suffer from a bradycardia, that is if a heart that beats too slow to fulfil the physiological needs of the patient. Intracardiac pacemakers apply electrical stimulation in the form of pulses to the heart in order to generate a physiologically appropriate heartrate. Alternative or additional functions of intracardiac devices comprise providing other electrical or electromagnetic signals to the heart or its surrounding tissue, sensing electrical or electromagnetic signals or other physiological parameters of the heart and/or its surrounding tissue.

Document U.S. Publication No. 2012/0172690 A1 discloses a leadless pacemaker device which comprises a multi-piece tine fixation subassembly with a set of four remotely deployable active fixation tines at its distal end. The document further describes a fixation of the pacemaker to a patient tissue using the four tines positioned in a circular arrangement during minimally invasive surgery. The tines are configured to penetrate into and out of a patient's tissue of the heart when deployed and positioned adjacent to this tissue in order to securely fix the device and to provide a mechanical contact of an electrode to the patient's tissue within the center of the circular arrangement of the active fixation tines. The known active fixation tines further provide a forward pressure of electrode onto tissue to assure good electric electrode-tissue contact. However, the state-of-the-art pacemaker with known header geometry takes up a substantial portion of the pacemaker's length resulting in a longer overall device length, which could be limiting in applications of smaller patient anatomy or atrial placement.

Conversely, with known solution headers integrated into a device and maintaining constant device size, the header design takes space from other critical components, such as the battery or electronics module, affecting higher criticality device features, such as device longevity or therapeutic features that could have been incorporated into a larger electronics module.

Accordingly, there is the need for implantable intracardiac devices having smaller dimensions and at the same time providing a robust attachment mechanism and low manufacturing effort and costs.

The present disclosure is directed toward overcoming one or more of the above-mentioned problems, though not necessarily limited to embodiments that do.

### SUMMARY

At least the above problem is solved by the header assembly for an implantable intracardiac device in claim 1 and the implantable intracardiac device defined in claim 10.

In particular, the header assembly for an implantable intracardiac device according to the present invention comprises an anchoring device that has at least one tine extending from a base ring, wherein the base ring is conically formed, i.e., has the shape of a truncated cone.

The header assembly is accommodated at the distal end of the intracardiac device as described in more detail below and provides a tine-based anchoring device or fixation comprising the at least one tine.

In one embodiment, the header assembly further comprises a header cap, a header spacer and optionally a collar. Particularly, the anchoring device is arranged between the header cap and the header spacer. The header spacer and/or the collar may have a washer-like shape, i.e., a flat hollow cylinder with a through opening having a smaller diameter than the cylinder wall. In one embodiment, the header cap is configured to receive and/or to retain a collar, particularly a washer-like collar, wherein particularly the collar is configured to elute an anti-inflammatory and/or immune-suppressive compound or composition, particularly towards the tissue of a patient that contacts the pacing electrode in a deployed state.

Particularly, the proximal end of the header assembly is formed by the header spacer, the base ring with the at least one tine extending therefrom, and the header cap and optionally the collar, wherein the mentioned elements may be arranged in this consecutive order from the proximal to the distal end of the header assembly. The header assembly may include a collar, particularly a washer-like collar, that is configured to elute an anti-inflammatory and/or immune-suppressive compound or composition, particularly towards the tissue that contacts the pacing electrode in a deployed state, and that is received or supported by the header cap.

In one embodiment, the header assembly comprises a plurality of tines, particularly three to six tines, e.g., four tines, extending from the base ring, wherein the tines preferably are evenly distributed along the whole circumference of the base ring of the anchoring device. In one embodiment, the base ring is conically formed in such way that the distal end of the base ring has a greater diameter compared with its proximal end. The axis of the base ring preferably extends parallel to the axis of a housing of the intracardiac device or, in other words, from the proximal to the distal direction. The at least one tine or the plurality of tines provides or is configured for the fixation of the device within the heart's tissue after deployment.

Particularly, the implantable intracardiac device, for example, a leadless pacemaker, has a cylindrical housing and the header assembly described above and in further embodiments below. Further, an electrode, particularly having preferably a rounded distal surface configured to contact the tissue of a patient, projects from the distal end of the housing, wherein the header assembly is arranged at and attached to the distal end of the housing of the intracardiac device such that the electrode projects through the header assembly, i.e., a respective through-going or complete opening of the header assembly. The electrode may act as a pacing electrode and/or sensing electrode for delivering and/or sensing therapeutic or physiological electrical pulses. The opening may be a central opening. The header spacer, the base ring of the anchoring device, the header cap, and optionally the collar comprise the opening, wherein the size of the opening of the spacer may be such that an electrode feedthrough or at least an electric conductor extending through the electrode feedthrough located at the proximal end of the electrode may be arranged within this opening. The cylindrical housing may comprise the electronics module having a processor and an energy source (e.g., a battery or coil (for wireless charging)) and, if applicable, a communication component such as an antenna. The processor may be adapted to process signals determined from the patient's body or received from the surrounding environment and/or to produce signals for treatment of the patient's heart. Such signals may comprise electrical stimulation in the form of pulses in order to generate a physiologically appropriate heartrate, and/or other electrical or electromagnetic signals to the heart or its surrounding tissue. Such signals are transformed and transmitted by the electronic module and may be applied by the electrode to the heart or its surrounding tissue. The electrode is electrically connected to the electronics module and the energy source. The hermetically sealed housing may comprise electrically conducting material, e.g., titanium, and may function as another electrode.

The above described header optimizes space due to the conical base ring and results in simpler and cheaper header components. In addition, the proposed header assembly allows an automated assembly, which results in less costs during manufacturing of the implantable intracardiac device. Accordingly, the axial length and volume of the header can be minimized with the header assembly according to the present invention. Particularly, the base ring is conically shaped to allow for axial height reduction. This improvement allows more space for other more critical features of the device, such as the battery, which would increase device longevity. The saved space may be also allotted to the electronics module, to incorporate more therapeutic features. Conversely, for the same battery and electronics module size, a reduction in header length would allow for a reduction in overall device length. This may enable an application for smaller patients, or alternate placement within the heart, such as the right atrium.

In one embodiment, the base ring has a plurality of recesses (i.e., material cutouts, slots or other geometry such as a meander structure comprising a plurality of curved portions), for example, at least two groups of two recesses arranged adjacent in circumferential direction within said base ring's distal end surface and extending from the distal side into the base ring's body. Alternatively, the plurality of recesses may extend from a proximal end surface of the base ring into the base ring body. As a further alternative, the plurality of recesses may extend both form the distal end surface and the proximal end surface of the base ring into the base ring body. In one embodiment, the length of one recess is at least ¼ of the length of the base ring (i.e., its dimension into the axial direction). In one embodiment, the length of one recess is maximum ⅔ of the length of the base ring. In another embodiment the length of the recess may be at least ½ of the length of the base ring. The plurality of recess allows for strain reduction during the shape setting process.

In one embodiment, each of the at least one tine comprises an abutting section directly extending from the base ring of the anchoring device and forming a connection with the base ring and a middle section, wherein the abutting section of the tine continues the conical form of the base ring. Each of the plurality of tines terminates into the base ring tangent to the arc of the tines just below the surface of the header cap, and the base ring is contained between the header cap and the header spacer. The middle section of each tine of the plurality of tines has a curved form (e.g., circular curved) and the end section furthest from the base ring comprises a straight section that may be bent outwardly or may run parallel to the housing. Other forms of each extending tine are possible, as well. The base ring and/or the at least one tine may partially or fully consist of biocompatible material, e.g., a shape-memory and/or super-elastic material, for example, nitinol.

Particularly, the base ring with the at least one tine is clamped and fixed between the header spacer (on its proximal side) and the header cap (on its distal side). In one embodiment, the header spacer comprises at least two projections or struts extending in distal direction, wherein an inner side face of each of the at least two projections form a positive connection with the conical base ring. Preferably, the inner side face of each of the at least two projections has the same inclination or slope as the outer lateral surface of the base ring and, if applicable, the at least two projections support the abutting section and optionally at least partly the middle section of each tine. The spacer prevents the base ring from making contact with the device housing or from damaging any Parylene or other coating material on the device housing. In one embodiment, each of the at least two projections of the spacer fits into a respective recess of the header cap.

The spacer as well as the header cap may comprise an electrically isolating material, for example, PEEK (polyether ether ketone).

In one embodiment, the base ring and the at least one tine are integrally formed in one-piece, wherein particularly, the base ring and the at least one tine are made or formed from a tube. Making or forming may involve laser cutting. In one embodiment, the base ring and the at least one tine are laser cut from a tube made of a super-elastic and/or shape-memory material, particularly nitinol. Particularly, after initial cutting, the base ring and the at least one tine are preliminary iso diametric (i.e., the at least one tine is straight), and following initial cutting, the anchoring device made of the super-elastic and/or shape-memory material may be shape set by heating then cooling while held in the desired shape, wherein during shape setting the at least one tine is curved and the base ring is conically formed.

In one embodiment, the header cap comprises a recess at its distal end face, wherein the recess is adapted to accommodate the collar. The header cap extends over the base ring between each tine to securely attach the base ring and thereby the at least one tine to the device housing. The header cap may comprise at least two slotted inserts, or other suitable geometric cutout at its distal side or surface, to allow for a tool interface during assembly, or the option of automated assembly. In one embodiment, the at least two slotted inserts are located within the recess for the accommodation of the collar.

In one embodiment, the header cap comprises at least two radially outward projecting rim flanges, particularly two partially circumferential rim flanges, to allow engagement with the device housing to prevent axial dislodgement of the header assembly from the device. The at least two rim flanges may form a fraction of the outer circumference of the header cap, for example, at its distal side.

In one embodiment, the distal end of the housing comprises a recess with at least two lateral projections (inwardly projecting rim flanges), wherein the header assembly is configured to be initially rotatable positioned within the recess and fixed by the lateral projections cooperating with respective lateral projections or the above described radially outward projecting flanges of the header cap. In other words, the outer diameter of the device housing extends past the electronics module portion and comprises at least two partially circumferential flanges projecting inwardly, allowing for engagement with the header cap. As indicated above, the header cap and the device housing comprise mating features that prevent rotational back twist once assembled.

In one embodiment, the collar comprises an electrically isolating material, for example, silicone, particularly so that the electrode protruding though the through-going opening is electrically isolated against the device housing and/or the at least one tine. The collar may be glued in place or retained within the recess of the distal end face of the header cap in order to securely fix the collar to the header cap. Furthermore, the collar preferably comprises one or more anti-inflammatory and/or immune-suppressive drugs, such as, for example, a dexamethasone compound such as dexamethasone sodium phosphate or dexamethasone acetate. In one embodiment, the collar comprises or essentially consists of a silicone matrix embedded with an anti-inflammatory and/or immune-suppressive compound, preferably a dexamethasone compound. Advantageously, the release rate of the anti-inflammatory and/or immune-suppressive may be controlled by the silicone matrix.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawing, wherein:

FIG. 1 shows an embodiment of the inventive intracardiac device with the inventive header assembly in a perspective, exploded side view,

FIGS. 2 and 3 show the embodiment of FIG. 1 in the assembled state, and

FIG. 4 shows another embodiment of the inventive intracardiac device with the inventive header assembly in a perspective view.

### DETAILED DESCRIPTION

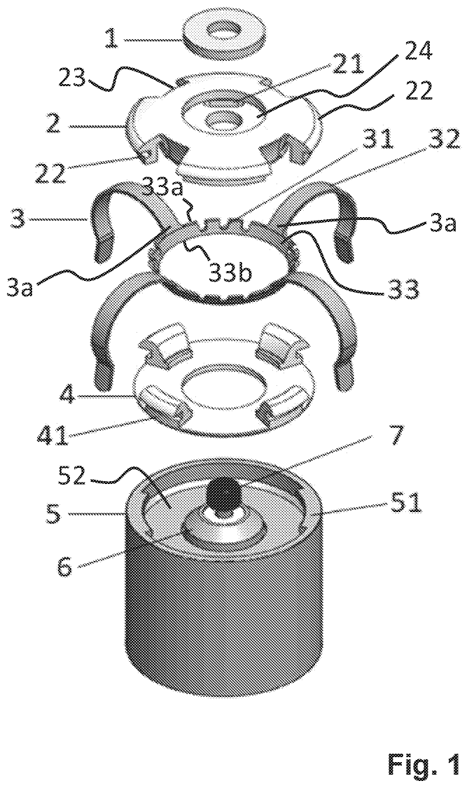

FIG. 1 illustrates an exploded view of the components of the implantable intracardiac device, e.g., a leadless pacemaker, with the header assembly. The components are a circular washer-like collar 1, a substantially flatly dome-shaped header cap 2, four tines 3 formed integrally with a base ring 33, a washer-like header spacer 4, a cylindrical device housing 5, and an electrode feedthrough 6 with an electrode 7 extending therefrom in distal direction and configured to act as a pacing and/or sensing electrode. The base ring 33 is conically formed in such way that a distal end 33a of the base ring 33 has a greater diameter compared with its proximal end 33b.

The header cap 2 is designed to retain the base ring 33 with the tines 3 and the header spacer 4 by locking them into the device housing 5 with a quarter turn, engaging radially projecting rim flanges 22 of the header cap 2 with the inner lateral projections (inner rim flanges) 51 at the device housing 5, particularly at the inner surface of a distal recess 52 at the device housing 5. The projections 51 allow space for the tines 3 to engage with the header cap 2.

The header cap 2 comprises recessed slots 21 to allow for tooling interface and allow for optional automation of assembly. Further, the header cap comprises a recess 24 at its distal side into which the collar 1 is assembled. The collar 1 may be glued within this recess 24.

The tines 3 extend from the conical shaped base ring 33 which comprises four groups of three material relief recesses (slots) 31, each extending from its distal side into the ring's body to allow for shape setting of the conical ring 33. The recesses 31 are disposed over the circumference of the base ring 33 and one of the recesses 31 is arranged adjacent to another one of the recesses 31. Each tine has an abutting section (flex zone) 3a, a curved middle section and optionally a straight end section (furthest from the base ring 33) kinked outwardly. The tines 3 provide the mechanical fixation of the intracardiac device within the patient's heart after deployment and penetration of the heart's tissue such that the central electrode 7 is in mechanical and electrical contact with the inner tissue of the patient's heart within one chamber.

In this embodiment of the design of the intracardiac device, the device housing 5 is coated with a parylene provided with a deposition process to isolate the body of the housing 5 from the electrode 7.

The header spacer 4 holds the tines 3 substantially tangent to the electrode 7 and ensures electric isolation between the tines 3 and the housing 5. The header spacer 4 is designed such that four projections 41 beneath each tine 3 are shaped (e.g., rounded) to create a smooth and tangent transition from the header assembly to the device housing 5. Each projection 41 supports with its inner surface one tine 3 at its flex zone providing a positive connection and each projection 41 extends from the distal side of the spacer 4. This reduces the propensity of scar tissue formation and tissue ingrowth by minimizing geometric discontinuities. The projections 41 are accommodated within respective recesses 23 of the header cap 2, such that the base ring 33 with the tines 3 is clamped and thereby fixed between the header cap 2 and the spacer 4. The recesses 23 extend laterally from the circumference of the header cap 2 into the body of the header cap 2.

The header cap 2 may comprise notches configured to receive the tines 2 of the anchoring device as depicted in FIGS. 1 to 3. Alternatively, the header cap 2 and the header space 4 together form a circumferential recess in the assembled state, in which the base ring 33 of the anchoring device is arranged and retain. Preferably, the header cap 2 and the header spacer 4 are configured to allow rotatable arrangement of the anchoring device. In other words, the anchoring device may be rotated in the assembled state.

The collar 1, the header cap 2, the base ring 33 and the spacer 4—each component comprises a central through-going opening for accommodation of the electrode 7. The diameter of the central opening of the spacer 4 is such that the electrode feedthrough 6 is located within this opening in the assembled state. The diameter of the electrode feedthrough 6 is greater than the diameter of the electrode 7.

The housing 5 of the intracardiac device contains a battery and an electronic module comprising a processor and ensures hermetically sealing of these components. These components are electrically connected to the electrode and provide the electrical stimulation of the heart or processing of electrical signals determined from the heart. Further, the housing may contain components for communication such as an antenna.

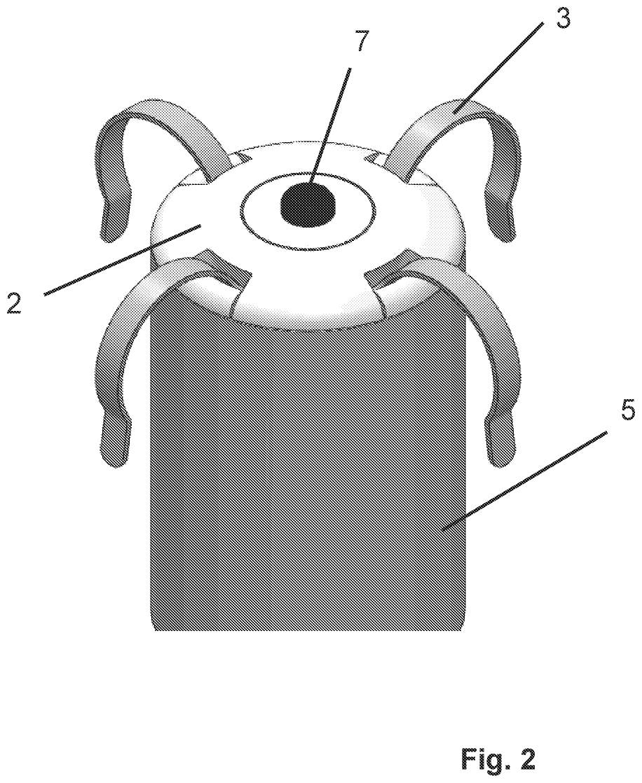

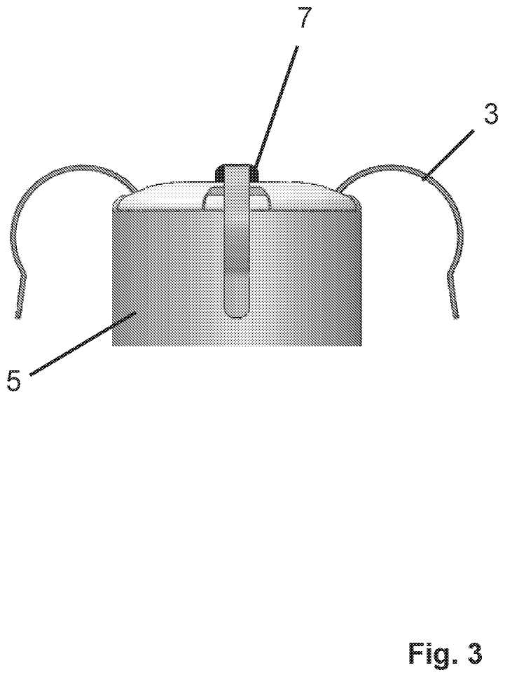

FIGS. 2, 3 show the intracardiac device with the header assembly according to the present invention in its assembled state in a perspective view and side view, respectively. Particularly FIG. 3 illustrates the novel and inventive flat design of the header assembly according to the present invention.

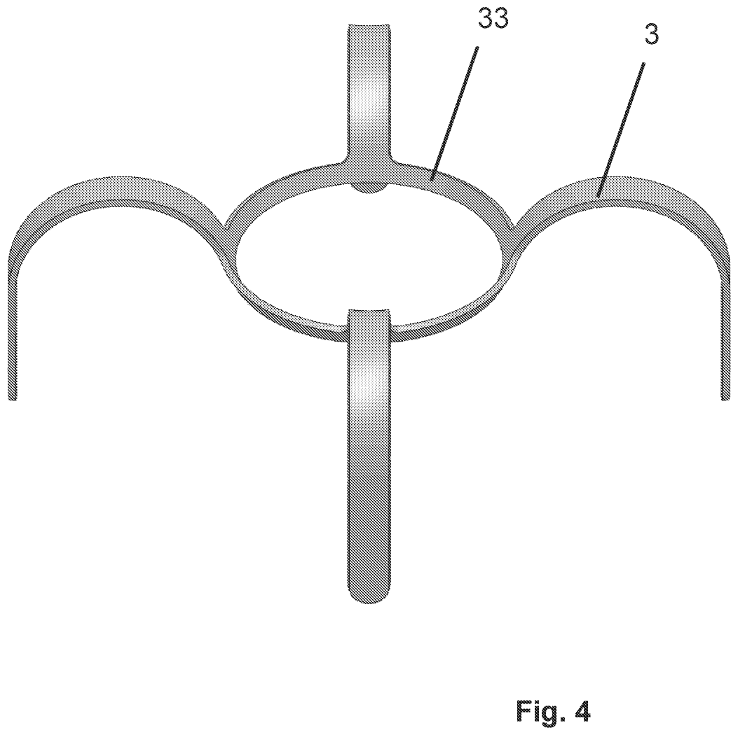

FIG. 4 illustrates an alternative but also preferred embodiment of the anchoring device according to the present invention. Therein, the anchoring device consists of a base ring 33 and four tines 3, particularly made of a super-elastic, shape-set material, such as nitinol, wherein preferably the base ring 33 and tines 3 are integrally formed in one-piece.

The base ring 33 is a coned-shaped, flat ribbon or sheet with a thickness being preferably equal to the thickness of the tines, from which each of the four tines 3 extends, respectively. The base ring 33 has the form of an inverted truncated, right circular, flat cone, wherein the lateral surface of the cone is inclined with respect to the rotatory axis of the cone and the anchoring device. The conical feature provides retention within the header that has been dramatically height reduced, to allow for size reduction of the overall device.

The four tines 3 particularly are evenly arranged at the base ring 33. Each of the tines 3 comprises a curved section, which extends from the base ring 33 along the inclination angle of the coned-shaped base ring 33, and a straight section extending from the curved section. The reduction in height of the conical base even further allows the shape setting to be successful without passing strain limits too high for the material.

The above intracardiac device with its inventive header assembly has reduced length in axial direction due to the conical shaped base ring 33. It is therefore less intrusive to the mechanics of the cardiac cycle and more suitable for smaller patients and challenging situations with regard to the heart (e.g., the accommodation within a heart's atrium).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A header assembly for an implantable intracardiac device comprising, an anchoring device having at least one tine extending from a base ring, wherein the base ring is conically formed.

2. The header assembly according to claim 1, wherein the base ring is conically formed in such way that a distal end of the base ring has a greater diameter compared with its proximal end.

3. The header assembly according to claim 1, wherein the base ring has a plurality of recesses, including at least two groups of two recesses arranged adjacent in circumferential direction within said base ring's distal end surface.

4. The header assembly according to claim 1, wherein each of the at least one tine comprises an abutting section directly extending from the base ring, wherein the abutting section of the tine continues the conical form of the base ring.

5. The header assembly according to claim 1, wherein the base ring and the at least one tine are formed integrally in one-piece.

6. The header assembly according to claim 5, wherein the base ring and the at least one tine are substantially made of a super-elastic and/or shape-memory material, comprising nitinol, wherein the anchoring device is cut from a tube made of the super-elastic and/or shape-memory material, and set into the desired shape after cutting.

7. The header assembly according to claim 1, further comprising a header cap, a header spacer and optionally a collar, wherein the anchoring device is arranged between the header cap and the header spacer.

8. The header assembly according to claim 7, wherein the header spacer comprises at least two projections extending in distal direction, wherein an inner side face of each of the at least two projections form a positive connection with the conical base ring.

9. The header assembly according to claim 8, wherein each of the at least two projections of the spacer fits into a respective recess of the header cap.

10. The header assembly according to claim 7, wherein the header cap comprises a recess at its distal end face, wherein the recess is configured to receive or retain the collar.

11. An implantable intracardiac device with a cylindrical housing and the header assembly according to claim 1, wherein an electrode projects from the distal end of the housing, wherein the header assembly is arranged at the distal end of the housing of the intracardiac device such that the electrode projects through the header assembly.

12. The implantable intracardiac device according to claim 11, wherein the distal end of the housing comprises a recess with at least two lateral projections, wherein the header assembly is adapted to be initially rotatable positioned within the recess and fixed by the lateral projections cooperating with respective lateral projections of the header cap.
